(19) United States
(12) Patent Application Publication
(10) Pub. No.: US 2005/0209627 A1
(43) Pub. Date: Sep. 22, 2005

(54) Expandable medical access device

(76) Inventors: George F. Kick, Casa Grande, AZ (US); Jay Lenker, Laguna Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/841,965

(22) Filed: May 7, 2004

Related U.S. Application Data
(60) Provisional application No. 60/554,334, filed on Mar. 18, 2004.

(51) Int. Cl. A61M 29/00

(52) U.S. Cl. 606/191

(57) ABSTRACT

In one embodiment, an access sheath includes a movable coil sheath that is insertable into a patient through an endoluminal or surgical approach. The sheath is inserted into a patient in a flattened configuration. A plurality of separate coils, elements, or hoops are connected by a fixed member and a control member, which are moved relative to each other to flatten the coils or hoops. Once inserted and advanced to the target surgical site, the sheath is selectively, and controllably, unflattened or expanded to a desired diameter or cross-section. A control member is affixed to one edge of the sheath and runs in the axial direction. By translating the control member in a direction parallel to the axis of the sheath, the operator causes the hoops or coils to rotate into a plane perpendicular or lateral to the axis of the sheath. A mechanical lock at the proximal end of the sheath permits the control member to be selectively constrained from translation and thus lock the sheath diameter in place.

### PRIORITY INFORMATION

This application claims the priority benefit under 35 U.S.C. § 119(e) of Provisional Application 60/554,334 filed Mar. 18, 2004.

### BACKGROUND OF THE INVENTION

1. Field of the Invention

This invention relates to medical access devices and, in particular, to expandable medical access devices for providing minimally invasive surgical access for various surgical procedures.

2. Description of the Related Art

Urethroscopy is procedure commonly used by urologist to explore the upper and lower urinary tract for diagnostic and therapeutic procedures and to provide access to the bladder and ureters. Visualization is often provided by a rigid or flexible scope (e.g., a cystoscope). In these cases, a sheath is often employed to protect the urethra during cystoscope placement as well as the placement of other instrumentation. Current sheaths use a “Dotter” configuration to advance a dilatation sheath over a guide catheter designed to track a guidewire under fluoroscopic guidance.

In some instances, strictures, or occlusions, are formed in the urethra because of disease or injury to the urothelium, the lining of the urethra. When a stricture occurs it can cause excessive restriction to urination, which must be relieved. A stricture may also prevent instrumented access to regions anatomically proximal to the stricture (e.g., the upper and lower urinary tract).

For the male patient, strictures are often encountered in the penis or prostatic urethra. Many surgical instruments are available to “break” or “release” the stricture so the initial procedure can continue. Opening of the stricture is often necessary because of the need for endoluminal access to a point anatomically proximal to the stricture in procedures such as Ureteroscopy. In addition, instrumentation developed for treating urological pathologies is generally too large to pass beyond a stricture. Accordingly, instruments have been developed to open urethral strictures. “Bougies”, “sounds”, or “urethrotomes” are examples of such instruments. A bougie is a tapered axially elongate dilator that expands the stricture. A bougie exerts a longitudinally directed force that, because of its tapered profile, radially dilates the urethra. A urethrotome is essentially a knife that cuts the stricture in a longitudinal direction. Often a bougie will not open a stricture because the scar tissue is so tough that dilation will not open it and a urethrotome is required for the opening procedure. Both procedures tend to cause trauma, which can lead to reformation of the stricture. Given the discomfort inherent in such procedures as well as the symptomatology leading up to such procedures, patients, as a rule, would like to avoid the consequence of renewed stricture formation.

Traditional cystoscopy uses cystoscopic sheaths, with effective diameters ranging from 2 to 12 mm (6 to 36 French, respectively), to gain endoluminal access to the urethra, bladder and ureters. Most procedures are performed through such sheaths. These sheaths are often oval in order to maximize the instrument carrying capability of the sheaths. The urethra is capable of easily carrying a sheath with a non-circular cross-section, as opposed to certain other body lumens such as arteries. The cystoscope sheath often includes a bulbous nose to aid in blunt expansion of the urethra during insertion. However, placing such sheaths through a urethra with a stricture often requires the use of dilators or a urethrotome as described above to open the stricture.

Since strictures preferentially occur on a side of the of the urethra, the passage of sheath with a round lateral cross-section may cause a selective shearing during intmemberuction, thus damaging the urothelium. If a round instrument if forcefully advanced, the stricture may be displaced laterally, again causing a stress disruption of the urothelium. In either case, formation of a subsequent stricture or occlusion is predictable sequelae of the procedure.

New devices and methods are needed to open strictures in such a way that trauma to the urethra is minimized. These devices need to provide for controllable tissue dilation once an initial tunnel is created. Such devices are particularly important for use in treating lesions of the bladder and the prostatic urethra, for example.

### SUMMARY OF THE INVENTION

There exists a general need for a ureteral dilator or temporary stent that can dilate with a more purely radially directed force and less longitudinal shear to the tissue than that exerted by traditional bougies or other dilators. There also exist a general need for an improved device for providing minimally evasive access for other surgical procedures.

Accordingly, in one embodiment of the invention, an expandable surgical access device comprises a tubular assembly that a lumen that extends along a longitudinal axis. The tubular assembly comprises a plurality of structural members and a tubular sleeve. A longitudinally extending first member is coupled to the structural members by connectors that permit rotational movement of the structural members with respect to the first member about an axis substantially perpendicular to the longitudinally axis but limit longitudinal movement of the structural members with respect to the first member. A second member is coupled to at least one of the structural members. An actuator is provided for moving the second member longitudinally respect to the first member. Longitudinal movement of the second member with respect to the first member causes the lateral structural members to rotate about the axis substantially perpendicular to the longitudinal axis of the access device. The structural members rotate between a reduced profile position in which the structural members are positioned substantially within a plane that is closer to being parallel to the longitudinal axis as compared to when the structural member is in an enlarged configuration.

Another embodiment of the present invention comprises a method of providing expandable surgical access. The method includes inserting a sheath into a body lumen, advancing the sheath to a target depth, expanding the sheath by rotating the ends of a structural element away from the longitudinal axis of the sheath; and removing the sheath from the body lumen.

In another embodiment of the invention, the tube-like expandable sheath device comprises a movable core consisting of a spring or plurality of circular components or elements such as, but not limited to, independent coils, having round, oval or other eccentric forms. The sheath further comprises a fixed, semi-rigid connector member extending the length of the device and having flexible or rotational attachment to each circular component. The fixed connector member has the properties of substantial column strength and tensile strength but is flexible and capable of bending in the lateral direction. The sheath further comprises a control member attached, in one embodiment, 180° opposed to the fixed connector member. Movement of the control member in a longitudinal direction relative to the axis of the sheath will shift the circular components from a position 90° or perpendicular to the fixed connector member shaft to a position nearing 0° or flat so that the longitudinal axis of the sheath is in the plane of the flat circular components. The inner and outer surfaces of the sheath may be thinly coated with a biocompatible material to present a smooth surface to tissue and instrument passage while allowing the control elements to change radial configurations of the circular components. In this embodiment, movement of the control member, arm or shaft will flatten the device to reduce the overall French size and/or vertical or cross-sectional profile under operator control to negotiate channels with asymmetric size and configuration. A control mechanism or handle and locking elements located at the proximal end of the device maintains the proper tension on the control member relative to the fixed member, to the extent needed to preserve the desired final configuration.

In an embodiment, the structural members have an oval cross-section, the sheath has a narrow outside diameter when the structural members are rotated into a plane parallel to the axis of the sheath. The sheath so configured would be capable of following a guidewire and allow passage through occlusions or strictures. By operating the control arm, the operator rotates the circular, or in this case oval, members into a plane perpendicular to the axis of the sheath. By rotating the circular members towards a position perpendicular to the axis of the sheath, the device outside diameter (OD) is increased and the sheath performs a radial dilation function to surrounding tissues. A corresponding increase in the inside diameter (ID) also occurs allowing the passage of a dilating balloon, optical scope or other devices such as surgical instruments and the like.

Another embodiment of the invention involves a method wherein additional dilation energy may be imparted to the sheath. In this method, a dilation balloon is placed where narrowing was observed either by external fluoroscopy, MRI, internal scope or a combination of the aforementioned. Once the balloon is positioned within the internal lumen of the sheath or channel where it passes through the stricture, the balloon would be inflated using fluids such as, but not limited to, radiopaque contrast media, saline, water, gas, or the like. The radial force imparted by the balloon could be applied to gently expand the lesion as well as the sheath. The expansion maneuver may be repeated over the length of the device as required by anatomic strictures. The method of balloon expansion may be repeated if the length of the balloon is not be as long as that of the sheath. Once a fully expanded, round cross-section sheath is formed by the combination of movable elements and radial force applied by balloon expansion, the balloon is deflated and removed. With the inner lumen or channel free of devices and strictures, procedures may be performed while the sheath protects anatomic surfaces outside the lumen. It is most common for a balloon to inflate to a circular or round cross-section although other cross-sectional configurations for the balloon such as oval, pear-shaped, bell-shaped, triangular, rectangular, or trapezoidal, are also possible.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

In addition, all of these embodiments are intended to be within the scope of the invention herein disclosed. These and other embodiments of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

FIG. 1A is a side perspective view of an exemplary embodiment of an access sheath in a reduced cross-sectional profile configuration;

FIG. 1B is a front view of a structural member of the access sheath of FIG. 1;

FIG. 1C is a side view of the structural member of FIG. 1B;

FIG. 2A is a side perspective view of the access sheath of FIG. 1 in an enlarged cross-sectional profile configuration;

FIG. 2B is a cross-sectional view taken through line 2B-2B of FIG. 2A;

FIG. 2C is a side view of an exemplary connector of the access sheath of FIG. 1A;

FIG. 3 is a side perspective view of a modified embodiment of the access sheath of FIG. 1 in a reduced cross-sectional profile configuration;

FIG. 4 is a side perspective view the sheath of FIG. 3 in an enlarged cross-sectional profile configuration;

FIG. 5 is a lateral cross-sectional view of the sheath of FIG. 3 and with a surgical instrument and an optical lens are inserted therethrough;

FIG. 6 is a lateral cross-sectional view of the sheath of FIG. 3 in a reduced cross-sectional profile configuration; and

FIG. 7 is a side perspective view of a modified embodiment of a sheath that utilizes a dilatation balloon.

### DETAILED DESCRIPTION OF THE PREFERRED EMBODIMENTS

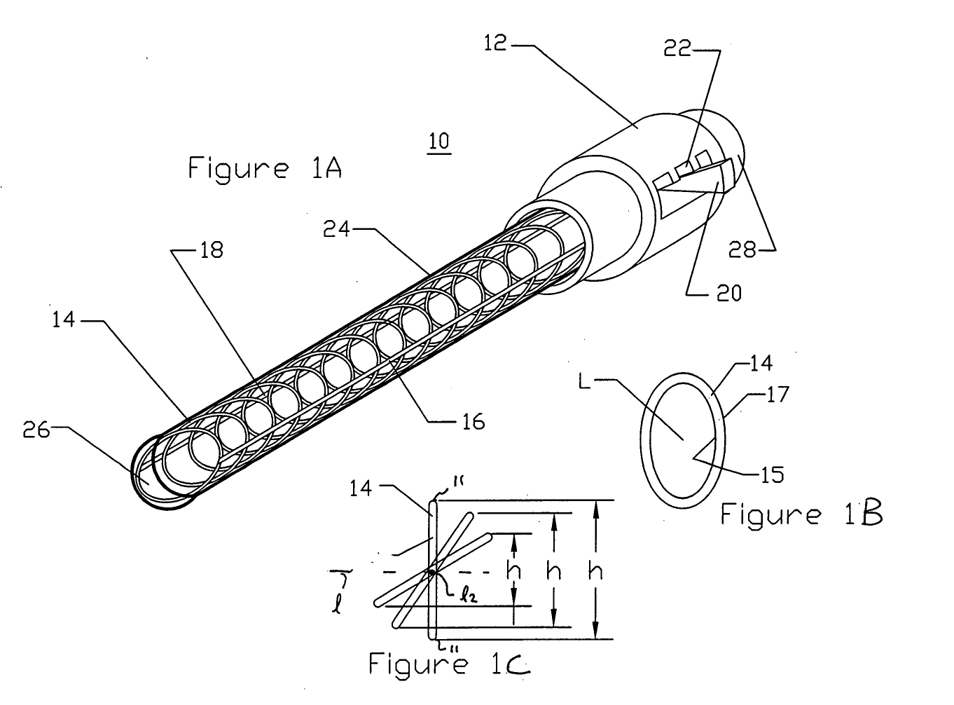

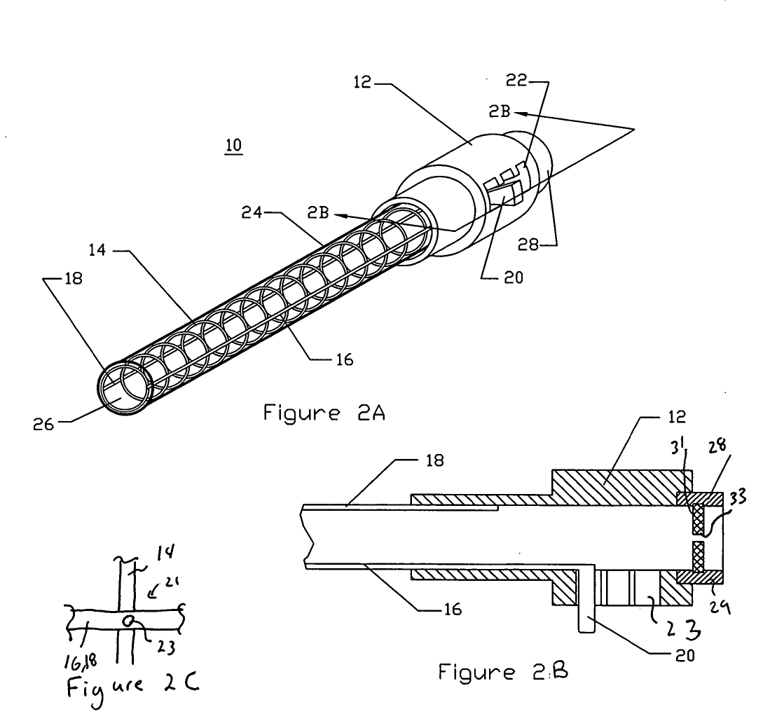

FIG. 1A illustrates a perspective view of an exemplary embodiment of an expandable access device 10 in a collapsed or reduced cross-sectional profile configuration. FIG. 2A illustrates the access device in a expanded or enlarged cross-sectional profile configuration.

In the illustrated embodiment, the access device 10 comprises a plurality of structural remembers or elements 14 an example of which is shown in FIG. 1B. Each lateral element 14 includes an inner surface 15 which defines at least in part an opening having a cross-sectional area and outer surface 17, which defines at least in part an outer profile. As shown in FIG. 1C, a longitudinal or axial axis l extends through the structural member 14 in a direction generally parallel to the longitudinal axis of the access device. As the structural element is rotated about a second axis l2, which is generally perpendicular to the longitudinal axis l, the height of the structural member is reduced as the width stays constant. When the structural element 14 is positioned in a plane substantially normal to the longitudinal axis of the device 10, the height is greatest. In contrast, as the structural element 14 is rotated such that it lies in a plane substantially parallel to the longitudinal axis l the height of is reduced. Accordingly, by rotating the ends 11 of the structural element 14 closer to the longitudinal axis l the profile of the structural member 14 is reduced and the access device 10 may be moved between collapsed or reduced cross-sectional profile configuration and the expanded or enlarged cross-sectional by rotating the structural elements 14 about the second axis l2.

With reference back to FIG. 1A, in the illustrated embodiment, the access device 10 also includes a hub 12, a control member 16 and a static member 18. As will be explained in more detail below, the control member 16 and static member 18 are configured for moving the structural elements 14 between the reduced cross-sectional profile configuration to the enlarged cross-sectional profile configuration. The device 10 preferably also further comprises a control knob 20 and a locking system 22 for selectively positioning the device 10 in a specific cross-sectional configuration.

A sleeve 24 is coupled to the external side 17 of the structural members 14 to define, in part, an inner lumen 26. In modified embodiments, the sleeve 24 may be coupled to the internal side 15 of the structural members 14. In still other embodiments, the structural members 14 may be embedded within the sleeve 24. In the figures, the sleeve 24 of the sheath 10 is rendered as transparent so that the internal components are visible. In addition, in the illustrated embodiment, the lateral elements 14 define a generally circular opening. However, as will be explained below, in other embodiments, the lateral elements 14 define openings with non-circular shapes.

With continued reference to FIG. 1A, the hub 12 serves as a point of stability and a grip for the sheath 10. The plurality of structural elements 14 are coupled to the control member 16 which runs generally along the longitudinal axis of the sheath 10. In the illustrated arrangement, the elements 14 are fixed, each at a point, to the control member 16. Preferably, the control member 16 is attached to the lateral elements 14 such that the structural elements 14 are constrained in the axial direction at specific positions on the control member 16 but some rotation of the structural elements 14 relative to the control member 16 is permitted. Any of a variety of connectors such as hinging elements (e.g., loops or pins through holes) and/or flexible material may comprise the attachment between the control member 16 and the lateral elements 14 as well as between the static member 18 and the lateral elements 14. For example, FIG. 2C illustrates a control member 16 or static member 18 that is attached to a structural member 14 by a connector 21 comprising a pivot shaft 23 that extend through but members 14, 16.

In the illustrated embodiment, the static member 18 runs generally along the longitudinal axis of the sheath 10 at a circumferential location on the lateral circular elements 14 that is preferably displaced substantially from that of the control member 16. The static member 18 is preferably affixed to the lateral elements 14 such that longitudinal relative motion between the static member 18 and the lateral elements 14 is prohibited while rotational relative motion is permitted. With reference to FIG. 2B, the proximal end of the static member 18 is preferably coupled to the hub 12 so that no longitudinal relative movement is permitted between the static member 18 and the hub 12. In contrast, the proximal end of the control member 16 extends within the hub 12 and is affixed to the control knob 20 so that the control member 16 moves with the control knob 20. The control knob 20 extends outside the hub 12 and is constrained to move within a slot 23 in the hub 12 so that longitudinal relative motion between the hub 12 and the control knob 20 is permitted within a specified range. In this configuration, the control knob 20 is fully moved to the proximal most position on the hub 12 so that the control member 16 has translated proximally and has rotated the circular lateral elements 14 about an axis perpendicular to the long axis of the sheath 10 into a collapsed configuration. The inner lumen 26 of the sheath 10 is substantially collapsed in this configuration, offering what is generally known as potential space.

In the illustrated embodiment, a locking mechanism 22 is formed by the hub 12 to permit selective fixed position control over the control knob 20. The hub 12 may include visual indicia (e.g., notches, numbers, etc.) that indicate the outside or inner diameter of the access sheath 10 as the control knob 20 is moved within the slot 23. In addition, as mentioned above, the locking mechanism 22 may include a plurality positions in between the distal most and proximal most position. Each position may be provided with visual indicia that indicate the diameter of the access sheath when the knob 20 is positioned at a specific position in the locking mechanism 22. Of course, those of skill in the art will recognize that other locking mechanism may be used, such as, for example, various combinations of latches, levers, locks etc. In this manner, in the illustrated embodiment, the device 10 may be selectively expanded to at least one cross-sectional profile and preferably a plurality of discrete intermediate cross-sectional profiles throughout a continuum between the largest and the smallest diameter and maintained at that size because of the locking mechanism 22.

With continued reference to FIG. 1, the sleeve 24 is preferably affixed at its proximal end to the hub 12. A seal 28 is preferably provided at the proximal end of the hub 12. The seal 28 is configured to provide an elastic seal between the hub 12 and any instrument inserted therethrough. As explained below, the seal 28 may prevent or reduce fluid loss when an instrument is inserted through the central lumen 26 of the sheath 10.

In modified embodiments, the static member 18 may also be moveable with respect to the hub 12. In such an embodiment, relative movement between the static member 18 and the control member 16 causes the structural elements 14 to rotate.

The structural elements 14, the control member 16 and the static member 18 may be fabricated from any of a variety of materials such as, but not limited to Elgiloy, nitinol, titanium, polytetrafluoroethylene (PTFE), stainless steel, polyamide, polyester, and the like. In the illustrated embodiment, the sleeve 24 is the primary body contact surface of the sheath 10 and is intended to form a continuous wall over the open framework of the lateral hoop elements 14. In such an embodiment, the external sleeve 24 may be fabricated from any of a variety of materials such as, but not limited to, PTFE, fluoroethylene polymer (FEP), silicone elastomer, thermoplastic elastomer (TPE), polyurethane, or the like. In certain embodiments, the external sleeve 24 may be fabric with openings or mesh grids, or it may be free from fenestrations and take the form of a continuous non-porous sheet. The external sleeve 24 may further be coated on one or both sides with anti-thrombogenic agents such as, but not limited to heparin, which is ionically, or covalently, bonded to the external sleeve 24. The external sleeve 24 may also be coated with antimicrobial agents such as, but not limited to, silver oxide, silver azide, betadine, or the like.

In still other embodiments, the external sleeve 24 may further be configured to carry electrical charge so that it can be used to deliver microwave or radio frequency (RF) energy, which can be used to cauterize or destroy cellular tissue. Metallic coatings with appropriate electrical connections leading out the proximal end of the sheath 10 may be applied to the surface of the external sleeve 24 for this purpose.

In a preferred embodiment, the external sleeve 24 is a creased, flattened axially elongate structure that expands to take on the general structure of the opened lateral elements 14. The external sleeve 24 is preferably fabricated from FEP, PTFE or expanded PTFE so that it comprises a low friction surface. Alternatively, the external sleeve 24 can be coated with materials such as, but not limited to, silicone oil, hydrophilic hydrogels, or the like to reduce friction with the tissues with which it comes in contact. In this preferred embodiment, the external sleeve 24 is not elastomeric. In a further embodiment, a plurality of longitudinally disposed structural members are arrayed around the lateral elements 14 to provide intermediate support so the containment layer 24 will not tend to cup or drape inwardly between opened lateral elements 14. The wall thickness of the external sleeve 24 ranges between about 0.0005 inches and about 0.100 inches and is often between about 0.005 inches and about 0.040 inches. The external sleeve 24 may be extruded or formed from flat sheet and welded or bonded to form a closed axially elongate cylinder of appropriate cross-sectional shape. The external sleeve 24 may form a containment layer to prevent the passage of fluids into or out of the sheath 10, a feature especially useful when working with malignant or carcinogenic tissue.

The working length of the sheath 10 is determined by the distance between the skin surface and the target surgical site. The sheath 10 may have a working length in the range from about 1 cm to about 175 cm and more typically range between about 5 cm and about 30 cm. The working length is generally the distance between the distal most edge of the external sleeve 24 and the distal end of the hub 12. The external sleeve 24 and the array or plurality of lateral elements 14, as well as the control member 16 and the static member 18 project axially into the inner lumen 26 of the hub 12 but this is generally not considered useable length for cannulating a patient. The radial strength of the sheath 10 formed by the structural elements 14, the control member 16, the static member 18 and/or the sheath in the described configuration desirably sufficient radial to expand most soft tissue in a uniform circular fashion. This radial strength may achieved by controlling the material, thickness and configuration of these components including the configuration orientation of the lateral members 14, which in themselves may be configured to have significant hoop strength, to create a structure that has resistance to hoop stress and point loads. In one embodiment, the distal edge of the sheath 10 is sharp in that the material of the tube is not edge treated in any way. The distal edge of the sheath 10 is, in another embodiment, atraumatic and not substantially sharp. In this embodiment, the distal edge of the sheath 10 is rendered blunt and atraumatic by attachment of a lateral member with large wall thickness and a rounded structural cross-section, or other technique known in the medical art.

It will be apparent from the disclosure herein that the access sheath 10 and/or the methods described herein may also find utility in a wide variety of diagnostic or therapeutic procedures that require an artificially created access tract. For such applications, the diameter of the tubular sheath 10 in the radially collapsed configuration and its expanded or enlarged configuration will depend upon the intended surgical application. For example, depending upon the application, the collapsed diameter of the sheath 10 may lie in the range from about 1 mm to about 10 millimeters. The expanded diameter of the sheath 10 may lie in the range from about 4 mm to about 50 mm. The wide variety of diagnostic or therapeutic procedures may include but are not limited to many urological applications (e.g., the removal of ureteral strictures and stones, the delivery of drugs, RF devices and radiation for cancer treatment, etc.), gastrointestinal applications (e.g., to the removal gallstones and appendix procedures, colon therapies, esophageal treatment and the treatment of bowel obstructions), cardiovascular applications (e.g., to provide access for minimally invasive heart bypass, valve replacement or the delivery of drugs or angiogenesis agents), vascular applications (e.g., minimally invasive access to the aorta or contralateral leg arteries for the treatment of, for example, an abdominal aortic aneurysm), gynecological applications (e.g., endometrial therapies, delivery of drugs, delivery of cancer agents, sterilization procedures, etc.), orthopedic applications and breast biopsies/lumpectomies

The static member 18 and the control member 16 are preferably fabricated from materials such as, but not limited, to polyester, polyamide, stainless steel, Elgiloy, nitinol, and the like. The control member 16 and the static member 18 are preferably sized so that their length extends to the distalmost structural element 14 and further extends into or to the hub 12. The diameters of the control member 16 and the static member 18 are preferably between 0.010 inches and 0.4 inches and more preferably between 0.30 inches and 0.25 inches.

The hub 12, the control knob 20, and the locking mechanism 22 are preferably fabricated from materials such as, but not limited to, Acrilonitrile Butadiene Styrene (ABS), polyvinyl chloride (PVC), polyethylene, polypropylene, and the like. As shown in FIG. 1, the control knob 16 is affixed coaxial with the hub 12. In this embodiment, the control knob 20 passes through a slot in the hub 12. The control knob 20 is constrained by the slot in the hub 12, which further has a plurality of notches or other locking mechanism 22. The control knob 20 may be spring biased to engage the notches, detents, or other locking mechanism 22. In another embodiment, the control knob 20 is manually guided into a locked position at user discretion with no bias. Gearing or other mechanisms may be also used to transmit the energy between the control knob 20 control member 16.

The locking mechanism 22 is, in the illustrated embodiment, a positive lock that is engaged to prevent, or disengaged to allow, relative rotation between the control knob 16 and the hub 12. In another embodiment, the lock 22 is a ratcheting mechanism that permits movement of the control knob 20 with binding or frictional click-stops. The proximal locking or ratchet mechanism 22 holds or maintains the diameter to that selected by the surgeon. The proximal lock assembly 22 is of small mass and profile to avoid interfering with surgical maneuvers. The lock 22 is further configured not require the attention of an assistant to maintain position or size. In another embodiment, an outer fixture may be placed at skin level and selectively affix to the hub 12 to assist with stabilization. The control knob 20 and the hub 12 are configured with no sharp edges or pinch points that could snare or perforate the glove of a surgeon and compromise sterility. As mentioned above, the locking mechanism 22 may also include visual indicia to indicate the diameter of the sheath 10 for a given position of the locking mechanism 22.

With reference to FIG. 2B, in the illustrated embodiment, the seal 28 comprises a housing that is fabricated from the same materials as those used to fabricate the hub 12. In one embodiment, the seal 28 further comprises an elastomeric membrane 31 that is suspended within the seal housing 29. The elastomeric membrane 31 is generally configured as a washer with a central orifice 33 capable of accepting instruments therethrough and sealing to the outer surface of said instruments. The central orifice 33 of the elastomeric membrane component of the seal 28 enlarges or shrinks as necessary to accommodate a wide range of instruments. The orifice diameter of the elastomeric membrane 31, in the unstretched state ranges from 0.1 inches to 1 inch. The elastomeric membrane 31 is fabricated from materials such as, but not limited to, silicone elastomer, thermoplastic elastomer, polyurethane, latex rubber, and the like. The elastomeric membrane 31 is preferably coated with a lubricant such as silicone oil or PTFE to minimize friction on passage of an instrument.

Provision for use of the sheath 10 over a guidewire is provided either by inserting the sheath 10 over the guidewire. In this embodiment, the guidewire is inserted through the central lumen 26f the radially collapsed sheath 10.

As mentioned above, FIG. 2 illustrates an oblique view of an expandable sheath 10 in its fully expanded configuration in which the sheath 10 has been expanded to a diameter larger than that shown in FIG. 1. The sheath 10 has been expanded to the maximum diameter allowable as determined by the cross-sectional shape of the structural elements or coils 14. The structural elements 14 may be expanded to any diameter through lumen 24 between the largest and the smallest effective diameter and maintained at that size under the influence of the locking mechanism 22. As the structural elements 14 are rotated more completely out of a plane substantially parallel of the longitudinal axis of the sheath 10, the cross-section of the inner lumen 26 moves from severely oval to oval with less difference between the major and minor axes to a limit condition of fully round when 90 degree rotation has been completed.

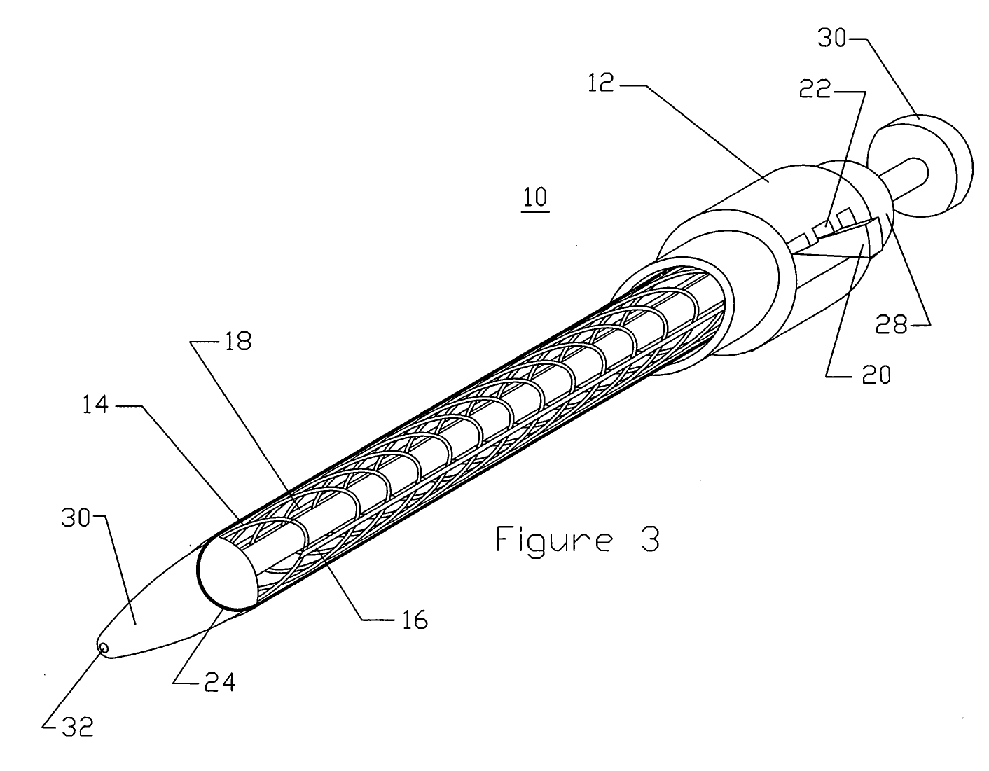

FIG. 3 illustrates an oblique view of a modified embodiment of an expandable sheath 10 in its collapsed configuration. The sheath 10 of FIG. 3 is fabricated using structural elements that have a cross-section diameter that is smaller in one direction (e.g., elliptical, pear-shaped, bell-shaped, or oval rather than circular), as are the lateral elements of the sheath in FIGS. 1 and 2. As mentioned above, it should be appreciated that in still other embodiments, the lateral elements may have other cross-sectional shapes (e.g., rectangular, triangular etc) the structural elements may also have one or more open sections in some embodiments.

As with the embodiment of FIGS. 1 and 2, the expandable sheath 10 comprises a hub 12, a plurality of elements 14, a control member 16, and a static member 18. The sheath 10 further comprises a control knob 20, a locking system 22, and an external sleeve 24. In this embodiment, the sheath 10 includes optional removable obturator 30, which further comprises an optional guidewire lumen 32.

Referring to FIG. 3, the obturator 30 is removable through the proximal end of the sheath 10 and is inserted to aid in introduction of the sheath 10 into the patient. The obturator 30 is typically used only in the radially compressed configuration of the sheath 10. The obturator 30 typically comprises a shaft, a distal tip and a proximal end. The shaft may be either metallic or polymeric in composition. The distal tip and the proximal end are most preferably fabricated from polymeric materials such as, but not limited to, ABS, PVC, polyurethane, PEEK, polypropylene, polyethylene, and the like. The shaft of the obturator 30 is preferably fabricated from metals or polymeric materials. The materials used for the manufacture of the obturator 30 include the same materials used for manufacture of the hub 12 and also include stainless steel, titanium, and other biocompatible metals. The obturator 30 assists in intmemberucing the sheath 10 into the lumen of the patient by helping to move tissue aside so that it is guided outward and over the exterior of the sheath 10. The sheath 10 is generally flexible although it could also be rigid and resist lateral bending. The obturator 30 is also able to be rigid or flexible depending on the needs of the physician in instrumenting the lumen of the patient. A flexible obturator 30 may be easily fabricated using a polymeric shaft or a segmented metal or polymeric shaft. In one embodiment, the sheath 10 is flexible but the obturator is rigid so that the sheath 10 with obturator is intmemberuced in a rigid configuration but is then able to become flexible once the obturator 30 is removed. A lateral cross-section of the obturator 30 will appear extremely oval or elliptical to match the internal lumen 26 of the sheath 10 when the lateral elements 14 are rotated nearly into the plane of the longitudinal axis of the sheath 10.

In a further embodiment, the obturator 30 comprises a guidewire lumen 32 that can track or follow a guidewire placed with a Seldinger method, for example. Such an embodiment of the expandable sheath 10 is especially useful for endovascular access cases.

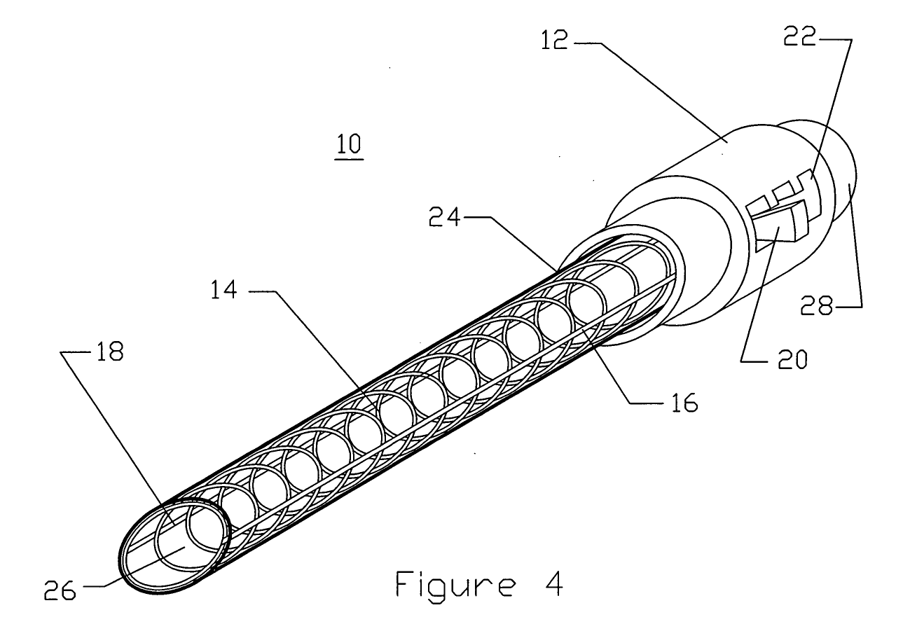

FIG. 4 illustrates an oblique view of the expandable sheath 10 of FIG. 3 in its fully expanded configuration in which sheath 10 has been expanded to a diameter larger than that shown in FIG. 3. Referring to FIG. 1 and FIG. 3, the oval structural elements 14 have the benefit of generating a reduced overall profile relative to circular structural elements 14. However, referring to FIGS. 2 and 4, the oval structural elements 14 are capable of generating a more useable internal lumen 26 capable of handling more than one instrument inserted through the sheath 10.

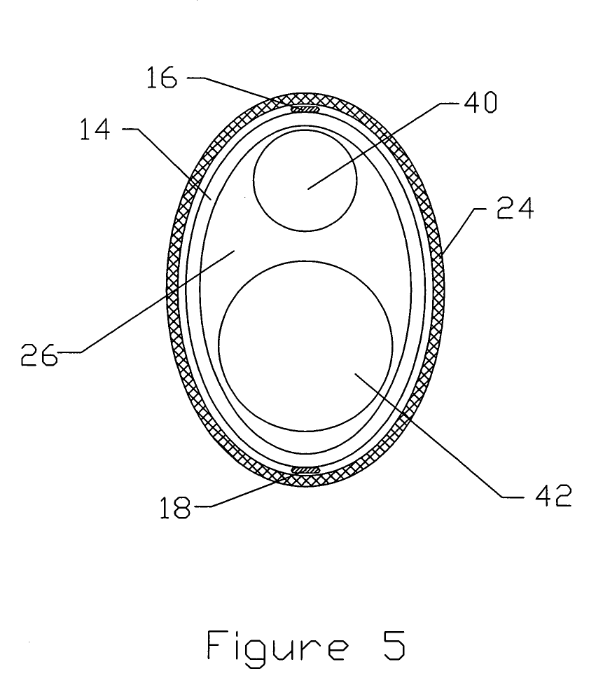

FIG. 5 illustrates a lateral cross-section of the working length of the sheath 10 of FIGS. 3 and 4. The sheath 10 is shown with its oval structural elements 14 rotated substantially away from the longitudinal axis of the sheath 10 to create a maximum or dilated configuration. The sheath 10 is further shown comprising an optical viewing lens or scope 40 and an instrument 42. The oval cross-section 26 of the sheath 10 allows for simultaneous passage of both the lens 40 and the instrument 42 through the lumen 26 of the sheath 10. The optical viewing lens or scope is typical of cystoscopes or other viewing devices and may be either rigid or flexible. Rigid scopes 40 typically have outer working length diameters in the range of 4 mm. The scope 40 and the instrument 42 are preferably passed through a seal 28 such as that shown in FIG. 4. The seal is an elastomeric element that prevents the escape or ingress of fluids between the instrument 42, the lens 40 and the sheath 10. The seal 28 of FIG. 4 may include central hole that accepts and seals to a single axially elongate smooth instrument. In another embodiment, the seal 28 has more than one hole so that more than one instrument can be inserted therethrough and seal to the seal 28. For example, the instrument 42 may be a set of graspers, which are inserted through a two-hole seal 28 along with a lens or scope to visualize the procedure. Such multiple instruments placed through a single sheath are an advantage of the expandable sheath 10. The large diameter, expanded expandable sheath 10 is capable of holding two or even more instruments. In yet another embodiment, the seal 28 is omitted to allow for a more direct surgical access to the sheath 10.

In the illustrated embodiment, the external sleeve 24 generally covers the outer extent of the structural members 14 but is not physically affixed to these lateral members. Accordingly, in this embodiment, the external sleeve 24 may remain stationary longitudinally relative to the hub 12 of the sheath while the structural members 14 rotate and move axially and radially relative to the external sleeve 24.

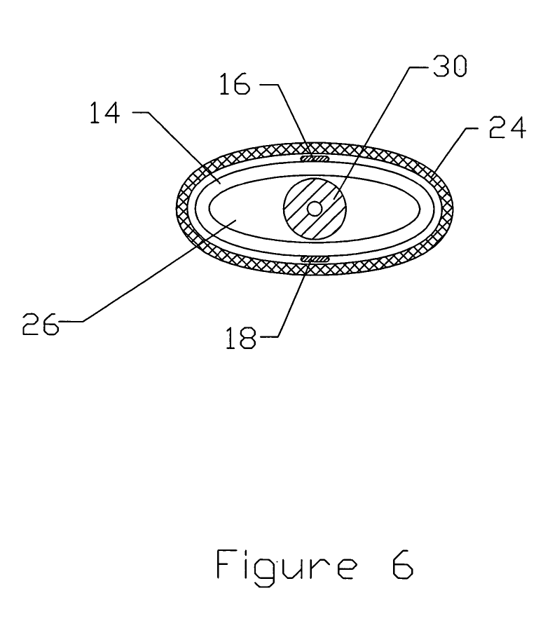

FIG. 6 illustrates a lateral cross-section of the working length of the sheath of FIG. 3 in a low profile configuration with its oval structural elements 14 rotated substantially toward the plane comprising the longitudinal axis of the sheath 10 thus creating a minimum or collapsed cross-sectional configuration. The sheath 10 is further shown comprising the shaft of an obturator 30 at that particular point in the cross-section. Note that the overall frontal or cross-sectional area of the working length of the sheath 10 is significantly less than that shown in FIG. 5 where the sheath 10 is expanded. The collapsed configuration is the preferred configuration for inserting the sheath 10 into a patient. The tip of the obturator 30 is generally larger than the shaft and is preferably designed to fill the available space within the internal lumen 26.

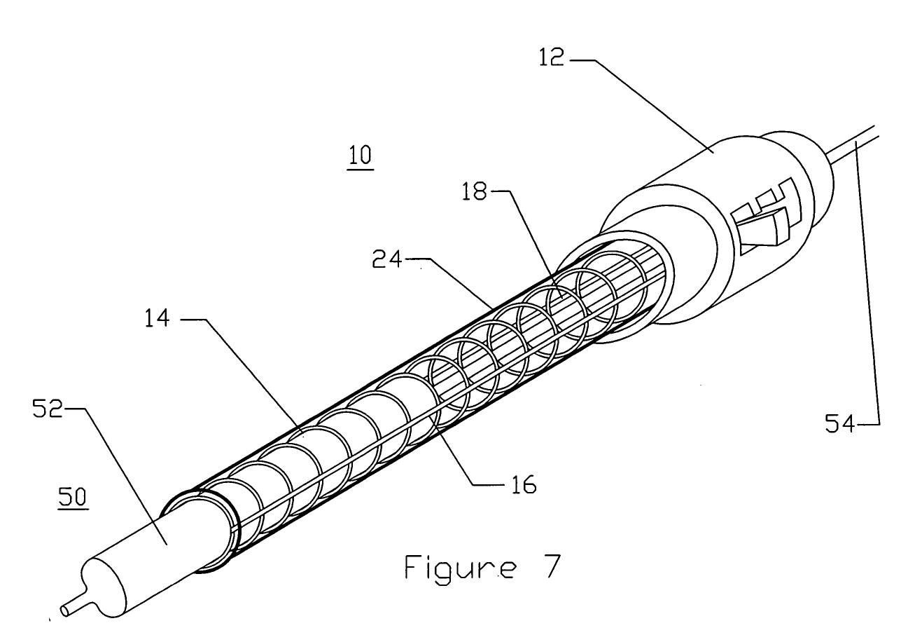

FIG. 7 illustrates a perspective view of another embodiment of the expandable sheath 10 that include oval structural elements 14 or elements of different shapes. In this embodiment, the cross-section of the working length of the sheath 10 is similar to that shown in FIG. 5 following rotation of the structural elements 14 into a plane normal to the longitudinal axis of the sheath 10. The oval structural elements 14 in this embodiment may be further dilated and formed generally circular or round by way of a dilatation device 50. The dilatation device 50 may comprise such devices such as angioplasty balloons, nitinol shape-memory elements that change shape above body temperature, and the like. In an embodiment, the oval structural elements 14 are malleable and take on a permanent round shape once dilated by the dilatation device 50. In the embodiment shown in FIG. 7, the dilatation device 50 comprises an inelastic balloon 52 and a catheter shaft 54. The catheter shaft passes through the hub 12 and the balloon 52 is positioned within the oval lateral elements 14. The balloon is inflated to cause the malleable oval structural elements 14 to become more circular in shape. The balloon 52 shown in FIG. 7 is located inside the distal half of the sheath 10. In FIG. 7, expanding of the structural elements 14 in the proximal end of the sheath has already taken place and the balloon 52 was deflated, moved to its current location and re-inflated. Materials used to make malleable lateral elements may include stainless steel, titanium, cobalt nickel alloys, and the like. In another embodiment, the structural elements 14 are shape memory nitinol and are martensitic at body temperature. Following dilation of the structural elements 14 with the dilatation means 50, the now expanded (e.g., rounded) lateral elements 14 may be heated using electrical resistive or Ohmic heating or even by infusion of warm fluids such as water into the sheath 10. Such heating is used to take the temperature of the structural elements 14 above a certain austenite finish temperature, at which point, they will return to their original oval shape which lends itself to reduced sheath 10 cross-sectional area, especially when the oval structural elements 14 are rotated to the collapsed position. The austenite finish temperature appropriate for such transition is preferably between 38 degrees centigrade and 42 degrees centigrade.

In certain embodiments, the sheath includes a guidewire channel, either through the sheath itself or through the center of a removable obturator. This guidewire channel provides the ability to insert the sheath, in its small diameter configuration, over a guidewire. In another embodiment, the sheath may be used as a probe under radiographic guidance (fluoroscopy, computer aided tomography (CAT), magnetic resonance imaging (MRI), or ultrasound). The sheath may further be inserted and manipulated under direct vision by including a small caliber scope to identify an anatomic path or features within a body cavity.

In one embodiment of use, once an initial tissue target is identified and the appropriate location of the access confirmed, the device could, under direct, precise operator control, enlarge the access lumen by applying radial force. The surrounding tissue applies a counter pressure to that exerted by the radially dilated device, which aids in maintaining stability of the device once it is expanded. The overall diameter of the sheath can be reduced, at operator discretion, by rotating a control knob, pushing or pulling a dilating control mechanism, or other control affixed to the control member that extends axially along one edge of the sheath. A lock on the control device further allows for selectively maintaining the configuration of the sheath, once set by the user.

In another embodiment, which is different than the passive mode of maintaining surgical retraction, the device can be intmemberuced via standard laparoscopic trocar and be selectively expanded to stabilize an organ or tissue with known, controlled circumvention pressure to provide the operator with a stable operative surface. Additionally, the device can be positioned to displace organs and structures to create a stable tunnel to expose a distant operative site.

Another embodiment, involves a method of use wherein the device is inserted as part of a system to capture an organ. The sheath is inserted to allow safe withdrawal of another device designed to contain the amputated organ or tissue, which can then be withdrawn through the sheath to a position outside that of skin level. This method conveys the benefit of laparoscopic surgery while avoiding the challenges associated with isolated removal of a diseased organ where malignant cell isolation is of a concern.

In accordance with another embodiment of use, a diseased organ or tissue mass is isolated by the surgeon by inserting the sheath to the target mass. An instrument can then be inserted through the sheath. These instruments may allow for various methods of cell or tissue destruction to be employed, with or without specimen removal. Access to the diseased organ may be accomplished under direct vision as part of a laparoscopic or percutaneous procedure. Exemplary uses of a sheath that may be selectively enlarged include applications in procedures to remove kidney stones, treat urethral strictures, perform biopsies or organ removal, perform stent implantation, and the like. The sheath is capable of being made smaller or larger in diameter to accommodate the size changes that are often required and sometimes unanticipated in a procedure. Following completion of the procedure, the sheath is removed from the patient, with or without the step of reducing the size of the sheath before removal.

In yet another embodiment, it is recognized that an insulating barrier placed on the outside, or inside, of the device would confine therapeutic or diagnostic cryogenic temperatures, radio frequency (RF) waves or microwaves so that they would not reach tissues surrounding the sheath. Instead of sustaining losses along the length of the sheath, these energies may be focused substantially on the tissue or organ targeted by the device at or near its distal end. In another embodiment, a seal layer is provided that prevents migration of fluids and other materials through the wall of the sheath. An insulating exterior or interior barrier that protects displaced, healthy tissue from destructive treatments being applied to diseased tissue within the confines of the device. Electrical, thermal and radiating therapeutic devices are incorporated herein. Tissue treated in this manner could be desiccated and rendered inert and of a reduced size for more easy removal. Furthermore, healthy tissue outside the sheath is protected against contamination by pathological tissue being removed or accessed by the sheath. Such protection of healthy tissue is especially important in the case of malignant or carcinogenic tissue being removed through the sheath so that potential spread of the disease is minimized.

As described above, the access sheath may comprise an axially elongate structure having a proximal end and a distal end. The axially elongate structure further has a longitudinal axis. As is commonly used in the art of medical devices, the proximal end of the device is that end that is closest to the user, typically a surgeon. The distal end of the device is that end is closest to the patient or is first inserted into the patient. A direction being described as being proximal to a certain landmark will be closer to the surgeon, along the longitudinal axis, and further from the patient than the specified landmark. A direction that is defined as being anatomically proximal is closer to the heart and further from the exterior of the patient. A direction that is defined as being anatomically distal is further from the heart and closer to the exterior of the patient. Anatomically proximal and distal are often the opposite of being proximal and distal as defined relative to a surgical or endoluminal instrument and are defined as such for the purposes of this disclosure.

The present invention may be embodied in other specific forms without departing from its spirit or essential characteristics. For example, the sheath 10 may include instruments affixed integrally to the interior central lumen 26, rather than being separately inserted, for performing therapeutic or diagnostic functions. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

It also should be noted that certain objects and advantages of the invention have been described above for the purpose of describing the invention and the advantages achieved over the prior art. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Moreover, although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. For example, it is contemplated that various combination or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, as mentioned above, it is intended that the scope of the present invention herein disclosed should not be limited by the particular disclosed embodiments described above, but should be determined only by a fair reading of the claims that follow.

1. An expandable surgical access device comprising:
A tubular assembly defining a lumen that extends along a longitudinal axis, the tubular assembly comprising a plurality of structural members and a tubular sleeve;
A longitudinally extending first member that is coupled to the structural members by connectors that permit rotational movement of the structural members with respect to the first member about an axis substantially perpendicular to the longitudinally axis but limit longitudinal movement of the structural members with respect to the first member,
A second member that is coupled to at least one of the structural members;
An actuator for moving the second member longitudinally respect to the first member;
Wherein longitudinal movement of the second member with respect to the first member causes the lateral structural members to rotate about the axis substantially perpendicular to the longitudinal axis of the access device, the structural members rotating between a reduced profile position in which the structural members are positioned substantially within a plane that is closer to being parallel to the longitudinal axis as compared to when the structural member is in an enlarged configuration.

2. The device of claim 1 wherein the sleeve is positioned on the outside of the structural members.

3. The device of claim 2 wherein the structural members are free to move longitudinally within the sleeve.

4. The device of claim 2, wherein the external sleeve is inelastic.

5. The device of claim 1 further comprising a hub coupled to the proximal end of the tubular member, the hub including a seal at the proximal end of the hub which prevents fluids from leaking out the proximal end of the hub after an instrument has been passed through the seal.

6. The device of claim 1 further comprising a lock to selectively position the first member relative to the second member.

7. The device of claim 1 further comprising a ratchet mechanism to selectively position the first member relative to the second member.

8. The device of claim 1 wherein the structural members have a circular shape.

9. The device of claim 1 wherein the structural members are non-circular in shape.

10. The device of claim 1 further comprising a removable obturator.

11. The device of claim 1 wherein the sheath is capable of bending out of the direction of the longitudinal axis of the sheath.

12. The apparatus of claim 1 further comprising shape memory lateral elements.

13. The apparatus of claim 1 wherein the structural members are circular in shape.

14. The apparatus of claim 1 further comprising an obturator having a guidewire lumen.

15. A method of providing expandable surgical access:
Inserting a sheath into a body lumen;
Advancing the sheath to a target depth;
Expanding the sheath by rotating the ends of a structural element away from the longitudinal axis of the sheath; and
Removing the sheath from the body lumen.

16. The method of claim 15, further comprising the step of inserting an obturator into the body lumen along with the sheath.

17. The method of claim 15, further comprising the step of inserting the sheath and an obturator over a guidewire.

18. The method of claim 15, further comprising the step of inserting instruments through a lumen of the sheath.

19. The method of claim 15, further comprising the step of reducing the diameter of the sheath prior to removal.

20. The method of claim 19, wherein the step of reducing the diameter of the sheath prior to removal comprises rotating the ends of the structural element towards the longitudinal axis.

21. The method of claim 15, further comprising deforming the structural members to further expand the sheath.

22. The method of claim 21, wherein the step of deforming the structural members to further expand the sheath comprises expanding a balloon position within the sheath.
